(19) United States
(12) Patent Application Publication
(10) Pub. No.: US 2008/0300501 A1
(43) Pub. Date: Dec. 4, 2008

(54) Fluid Analyser

(75) Inventors: Nicolaas Petrus Willard, Eindhoven (NL); Sepas Setayesh, Eindhoven (FR); Dagobert Michel De Leeuw, Eindhoven (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/995,698

(22) PCT Filed: Jul. 6, 2006
(86) PCT No.: PCT/IB2006/052282

§ 371 Date: Aug. 6, 2008

(30) Foreign Application Priority Data
Jul. 19, 2005 (EP) 05300601.1

(51) Int. Cl. A61B 5/08; H01L 51/05

(52) U.S. Cl. 600/532; 257/40; 257/E51.005

(57) ABSTRACT

A gas analyser (12) comprises a transistor (1) that has a cavity (7) between its gate (2) and its organic semiconductor (6) based conducting channel. In operation a component from a gas sample introduced into the cavity (7) may absorb onto an exposed absorption sensitive surface portion of the organic semiconductor (6). A detector (13) detects a change in the threshold voltage of the transistor caused by the component absorbing on the exposed surface portion. In response to detecting this change, the detector generates a measurement signal indicative of a concentration of the component in the sample.

The present invention relates to a fluid analyser and in particular a fluid analyser comprising a transistor.

There are many types of transistor devices that have been developed for diverse applications. Some known transistors have been used to detect and measure the concentration of volatile compounds in ambient air or exhaled breath. A sensor comprising one such transistor is described in, “Electronic noises, principles and application, J W Gardner, P N Bartlett, Oxford University Press, pp 101, 1999”. The sensor described therein detects volatile compounds by measuring a change in the work function of the transistor's gate after the volatile compounds absorb onto the gate. The transistor incorporates inorganic silicon based material, which is not itself sensitive to the presence of volatile compounds. These sensors have a limited sensitivity and the transistor's gate is a suspended metal gate that is expensive and difficult to construct. In “Handbook of Conducting Polymers, ed. TA Skotheim, R L Elsenbaumer, JR Reynolds, Marcel Dekker, New York, pp. 963, (1998)”, there is described a transistor that utilises an organic semi-conductor to sense gases. The electronic properties of such organic semi-conductors change as gases absorb on them, allowing the gases to be detected. This transistor comprises a common gate silicon wafer, a gate insulator, a drain and a source. The channel between the drain and the source comprises an organic semiconductor, one face of which forms an interface with the gate insulator. On its opposite face, the organic semiconductor forms an air interface. As gases absorb onto organic semi conductor at the semi-conductor/air interface, changes in the electronic properties of the semi-conductor occur that allow the absorbates to be sensed. It is believed that gas sensor's comprising transistors having this arrangement are still relatively insensitive.

There are several bio-markers in exhaled breath that can be used to detect or control diseases. Exhaled breath analysis is a non-invasive diagnosis or medication method that may be used by patients themselves at home to monitor their health. Patients are provided with a suitable breath analyser for their needs. One of the most widespread uses of breath analyses is detect the presence of NO in exhaled breath, the concentration of which in breath may be correlated with the severity of a patient's asthma.

There is a need for a fluid sensor that is relatively simple, non-expensive and sensitive.

Embodiments of the present invention aims to alleviate the above-mentioned problems.

According to the present invention there is provided a fluid analyser comprising: a transistor comprising, a gate and a semiconductor conducting channel, wherein the transistor defines a cavity between the gate and the semiconductor conducting channel such that in use, a component from a fluid sample introduced into the cavity may absorb onto an exposed surface portion of the semiconductor; and a detector for detecting a change in a property of the transistor caused by the component absorbing on the exposed surface of the semiconductor and in response thereto generating a measurement signal indicative of a concentration of the component in the sample.

In an exemplary embodiment, the analyser is a gas analyser and the semiconductor conducting channel is an organic semiconductor sensitive to the absorption of biomarkers.

In an exemplary embodiment, the property of the transistor is a threshold voltage.

There is also provided a method of analyzing a fluid sample, the method comprising; receiving a fluid sample into a cavity defined in a transistor between a gate of the transistor and a semiconductor layer that forms a conducting channel of the transistor, such that a component of the fluid can absorb on an exposed surface portion of the semiconductor layer; detecting a change in a characteristic of the transistor induced by the component absorbing on the exposed surface portion and in response thereto generating a signal indicating a concentration of the component in the sample.

An embodiment of the invention will now be described by way of example only with reference to the accompanying drawings in which:

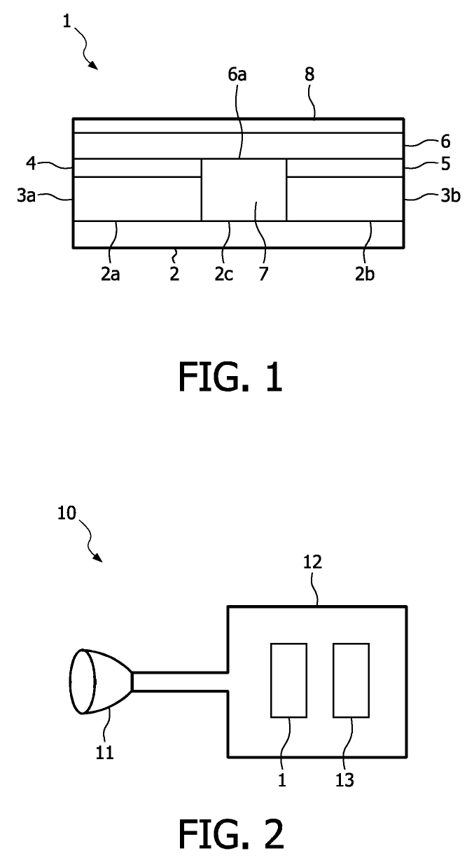

FIG. 1 is a schematic diagram of a transistor;

FIG. 2 is a schematic diagram of a fluid sensor comprising the transistor illustrated in FIG. 1.

Referring now to FIG. 1, which illustrates a field effect transistor 1 (FET). The FET 1 comprises a gate 2 typically comprised of heavily doped silicon wafer. Insulator material 3, which may be silicon oxide, covers a first portion 2a of a surface of the gate 2 forming a first insulator region 3a and on a second portion 2b of the surface of the gate 2 forming a second insulator region 3b, such that a gap in the insulator material extends across an exposed third portion 2c of the surface of the gate 2. In some embodiments a metal layer (not illustrated), typically gold is deposited on the third portion 2c to form an electrical contact.

If it is comprised of silicon oxide, the insulator layer 3 may be deposited to a thickness in the range of 100 to 300 nm, and preferably around 200 nm. An insulator layer 3 comprising silicon oxide may be thermally grown, and the gap generated by photolithography and etching.

In alternative embodiments, the insulator material 3 may comprise an organic polymer or a photolacquer. If the insulator layer 3 comprises an organic polymer the gap between the first region 3a and the second region 3b may be formed by moulding and the insulator layer 3 may be deposited to height of several microns. If the insulator layer comprises a photolaquer, the gap may be formed by exposure to ultra violet radiation and development of the exposed areas.

A source electrode 4, typically gold, is deposited on the first region 3a of insulator material and a drain electrode 5, also typically gold is deposited on the second region 3b of insulator material. The source 4 and drain 5 electrodes have a typical thickness of around 20 nm.

A layer of semiconductor material 6, in a preferred embodiment an organic semiconductor, extends from the source electrode 4 to the drain electrode 5 bridging the exposed third portion 2c of the surface of the gate 2. Thus, the gate 2, the insulator regions 3a and 3b, the source electrode 4, the drain electrode 5 and the semiconductor layer 6 define an air cavity 7 in which the exposed third portion 2c of the surface of the gate 2 faces an exposed surface region 6a of the semiconductor layer 6. Finally, a protective layer of foil 8 (typically comprising a polyimide, a polyester, a polycarbonate or the like) caps the organic semiconductor layer 6.

As will be appreciated by those skilled in the art, the FET 1 may be constructed using known semiconductor device fabrication techniques and the cavity 7 filled with clean air or an inert gas such as dry nitrogen.

In effect, the gas cavity 7 forms a dielectric between the gate 2 and the semiconductor layer 6. In operation, the FET's conducting channel runs through the semiconductor layer 6 between the drain 5 and the source 4 near the interface of the layer 6 and the cavity 7.

This interface between the semiconductor layer 6 and the cavity 7 enables the transistor to function as an effective gas sensor. Clean air samples can be introduced into the air or inert gas cavity 7 without affecting the dielectric properties of the cavity 7 and the electronic properties of the FET 1. However, volatile species in air or in exhaled breath introduced into the cavity 7 can influence the dielectric properties of the cavity 7 and the electronic properties of the OFET 1. More specifically, such volatile species absorb onto the exposed surface region 6a of the semiconductor layer 6, where they are close to the FET's conducting channel to strongly interact with it. It is these interactions that influence the electronic properties of the transistor, for example, its threshold voltage.

For a suitably calibrated FET 1, a measurement of the change in the threshold voltage caused by a particular component, for example NO, absorbed at the semiconductor/air interface, indicates the partial pressure (or concentration) of that species in the cavity 7. The selection of the material that comprises the semi conductor layer 6 depends upon the particular gas component that the OFET 1 is designed to detect. For example, certain organic semiconductors, including those based on polyarylamines, are very sensitive to NO absorption and are reactive with NO. Such organic semiconductors are ideal for use as the semiconductor layer 6 in an OFET 1 used in a NO detector. For good sensitivity, preferably, an organic semiconductor layer 6 has a thickness in the range 5 nm to 5 microns, and within a most preferred range of 30 to 100 nm. By using an appropriate material for semiconductor layer 6, embodiments of the invention may be used to sense other Bio Markers as well as NO, for example, acetone, ethanol, carbon monoxide and isoprene, as well.

A organic FET 1 comprising an organic semiconductor layer 6 may easily be constructed by first forming the gate 2, the insulator layer 3 and the source 4 and drain electrodes 5 using standard techniques. To complete the FET 1, a polymer foil 8 may be coated with an organic semiconductor layer 6, for example polyarylamine. This flexible double layer of polymer foil 8 and organic semiconductor 6 may then be placed so that the organic semiconductor layer 6 is brought into contact with the source 4 and drain electrodes 5 as shown in FIG. 1.

In a preferred embodiment, the absorption surface of the semiconductor layer 6 is relatively smooth, with a roughness of no more than a Ra of 50 nm and preferably a roughness with a Ra of 5 nm.

Systems embodying the invention may detect the presence of relatively low concentrations of volatile compounds in air or exhaled breath. For example, patients with asthma exhale NO in the range of 20 to 100 parts per billion (ppb) (as opposed to the 0 to 20 ppb of non-asthma sufferers), a concentration range that is detectable by the OFET 1.

The underlying principle that governs why absorbates influence the electrical properties of the transistor, including the threshold voltage, is not known. The influence may result from the absorbates creating new dopants in the organic semiconductor layer or the absorbates acting as interfacial dipoles.

In the above described example, for good measurement sensitivity, the width of the cavity 7 is preferably within the range of 0.5 microns to 500 microns, and most preferably around 10 microns.

In the above described example, there is a gap in the insulator layer 3 that forms part of the cavity 7. This is not essential. In alternative embodiments, the insulator layer may extend entirely across the gate 2. In such embodiments, the cavity 7 is defined by the insulator layer 3, the semiconductor layer 6 and the source 4 and drain 5 electrodes. In such embodiments, the height of the cavity 7 is determined by the height or the thickness of the drain 4 and source 5 electrodes, and is preferably more than 20 nm. In embodiments where there is a gap in the insulator layer 3, the height of the cavity 7 is mainly determined by the thickness of the insulator layer 3, typically around 200 nm for silicon oxide insulator and up to several microns for an organic polymer insulator layer.

Referring now to FIG. 2 of the drawings there is illustrated a breath gas analyser 10 embodying the present invention, which is suitable for use a home health care kit for asthma detection. The analyser 10 comprises a mouth piece 11 connected to a gas sensor unit 13. The gas sensor unit 12 comprises an OFET 1, as described above with respect to FIG. 1, and a detector and control circuit 12.

In use, a patient exhales breath into the mouthpiece 1 and the mouthpiece guides a breath sample to the cavity 7. The mouthpiece 1 is arranged to guide the sample to the cavity 7 at a controlled flow rate and temperature for the measurement to take place. The breath sample 7 passes through the cavity 7 allowing NO molecules in the sample to adsorb at the organic semiconductor/air interface. The detector and control circuit 12 measures any change in the threshold voltage or other electrical properties of the OFET 1 caused by NO adsorption and in response outputs a signal (not shown) indicative of the concentration of NO in the sample.

Although the embodiments described above relate to gas analysers, it will be understood that embodiments of the invention may be used to analyse other fluids, for example liquids.

Having thus described the present invention by reference to a preferred embodiment it is to be well understood that the embodiment in question is exemplary only and that modifications and variations such as will occur to those possessed of appropriate knowledge and skills may be made without departure from the spirit and scope of the invention as set forth in the appended claims and equivalents thereof. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements.

1. Fluid analyser comprising:
a transistor comprising, a gate and a semiconductor conducting channel, wherein the transistor defines a cavity between the gate and the semiconductor conducting channel such that in use, a component from a fluid sample introduced into the cavity may absorb onto an exposed surface portion of the semiconductor; and
a detector for detecting a change in a property of the transistor caused by the component absorbing on the exposed surface of the semiconductor and in response thereto generating a measurement signal indicative of a concentration of the component in the sample.

2. Fluid analyser according to claim 1, wherein the semiconductor conducting channel is an organic semiconductor.

3. Fluid analyser according to claim 2 wherein the organic semiconductor comprises polyarylamine.

4. Fluid analyser according to claim 1 wherein the transistor further comprises an insulating layer formed on a surface of the gate, the insulating layer leaving exposed a surface portion of the gate and wherein the gate, the semiconductor conducting channel and the insulating layer together substantially define the cavity with the exposed surface portion of the semiconductor facing the exposed surface portion of the gate.

5. Fluid analyser according to claim 4 wherein the transistor further comprises a source formed between a first portion of the insulator layer and the semiconductor and a drain formed between a second portion of the insulator layer and the semiconductor.

6. Fluid analyser according to claim 1 wherein the transistor further comprises a protective layer formed on an upper surface of the semiconductor.

7. Fluid analyser according to claim 1, wherein the transistor property is its threshold voltage.

8. Fluid analyser according to claim 1 wherein the fluid sample is a breath sample, the analyser further comprising a mouth piece for delivering the breath sample to the cavity.

9. Fluid analyser according to claim 1, wherein the component is one of: nitrogen monoxide, acetone, ethanol, carbon monoxide and isoprene.

10. A method of analyzing a fluid sample, the method comprising;
receiving a fluid sample into a cavity defined in a transistor between a gate of the transistor and a semiconductor layer that forms a conducting channel of the transistor,
such that a component of the fluid sample can absorb on an exposed surface portion of the semiconductor layer;
detecting a change in a characteristic of the transistor induced by the component absorbing on the exposed surface portion and in response thereto generating a signal indicating a concentration of the component in the sample.
